(19) United States
(12) Patent Application Publication
(10) Pub. No.: US 2018/0292388 A1
(43) Pub. Date: Oct. 11, 2018

(54) Methods for Establishing Threshold Limits for a Chemical or Biological Agent in a Target Species

(71) Applicant: Coyne IP Holdings, LLC, Atlanta, GA (US)

(72) Inventors: Shawn T. Coyne, Smyrna, GA (US); Kevin P. Coyne, Atlanta, GA (US)

(21) Appl. No.: 15/578,024

(22) PCT Filed: Jun. 3, 2016
(86) PCT No.: PCT/US16/35678

§ 371 Date: Nov. 29, 2017

Related U.S. Application Data
(60) Provisional application No. 62/170,486, filed on Jun. 3, 2015.

(51) Int. Cl. G01N 33/50

(52) U.S. Cl. CPC G01N 33/5014; G01N 33/5073

(57) ABSTRACT

Methods are provided for establishing a threshold exposure limit to a chemical or biological agent for the members of a target species taking into account the potential for variation in responses among the members of that species. Exemplary exposures include maximum safe exposure to a toxic agent, or minimum effective dose for a pharmaceutical. The method is particularly useful under circumstances wherein variation in responsiveness to the agent cannot be ascertained by directly exposing members of the target species to the agent.

### FIELD

The present application relates to the fields of drug development and analysis of chemical exposures. In particular, the application relates to methods for determining threshold limits, such as dose, exposure, or concentration limits, for exposure to a chemical or biological agent.

### BACKGROUND OF THE INVENTION

Humans and other species react to exposures of a wide variety of chemical and biological agents. These reactions may be beneficial or harmful, depending on the agent. Further, for many agents, the degree of reaction is usually positively correlated to the degree of exposure.

However, individual members of a species vary in their degree of response as a result of multiple factors including genetic variations among the members, their age, and the degree to which they have been exposed to certain environmental factors during their lifetimes. Therefore, there is no single dose or exposure that represents a “threshold” limit for all members of that species.

In some species—such as humans—scientific or ethical barriers may prevent a researcher from determining certain threshold limits for a population (or even a sample of a population) through direct experimentation on live members of that species. In such cases, the dose is often estimated from information gathered from other species in a process called dose extrapolation. For example, a common practice for toxicity testing during drug development and in industrial chemical exposure regulation is to conduct animal experiments, which, in the case of drug development, occurs prior to human clinical trials. Additionally, veterinarians often conduct small animal experiments to estimate doses for treatment of larger animal species.

The currently available procedure involves testing one or multiple animals within a species or multiple species to establish particular threshold dosages. Different measures can be utilized as a threshold dose in a test species, such as, for example, the No Observable Adverse Effect Level (NOAEL) measure, which is the highest observed dose that fails to cause a significant increase in adverse effects in a particular species when compared to an untreated control group. Additionally, the Minimally Effective Dose (MED) measure is the lowest dose that can produce a significantly beneficial effect in a particular species compared to an untreated control group. Test species routinely chosen to establish threshold dosages include, but are not limited to, species determined by their sensitivity to the biological or chemical agent or the species' biological relevance to the target species in terms of the particular agent's action. In other words, a test species is chosen that is expected to have a reaction to an agent, either beneficial or detrimental, similar to the anticipated reaction by a human or target animal. For example, rats, dogs or primates are typical test species for ultimate exposure of the same agent to humans or horses.

During threshold determination, an animal or series of animals from the test species are exposed to increasing or decreasing doses. A tested dose, or a dose mathematically chosen from a plurality of doses, is then established by a researcher to be the threshold dose in that particular species. The researcher then conducts analysis to convert the dose in the test species to an equivalent dose in the target species using known methods of dose extrapolation, or scaling. Examples include, but are not limited to: allometric scaling on the basis of body surface area, and pharmacokinetically guided dose extrapolation, utilizing clearance and volume of distribution.

Explicitly or implicitly, the extrapolated dose is associated with the presumed response of a representative (or reference standard) member of the target species, which may be defined as the median, mean, mode, or some other measure of centrality. (For example, when extrapolating doses from animals to humans, the reference standard human is often assumed to be a male adult weighing 60 kilograms.) This reference standard of the target species may also be associated with the minimum, maximum, or any hierarchical response of the species, as well as a centrality or hierarchical response of a plurality within the species.

Current practice often calls for an adjustment factor to be applied to accommodate the fact that not all members of the species may react identically. Often, the adjustment factor is simplistic and may be arbitrary. For example, the current practice in industrial chemical analysis and first-in-human clinical trial design is to apply a safety margin, often “10×” (meaning that the dose is reduced by a factor of 10) to the calculated threshold dose, termed “Human Equivalent Dose (HED)” to obtain the Maximum Recommended Starting Dose (MRSD). In this example of safety testing, the adjustment factor may be raised if there is reason to suspect increased toxicity in the target species as a whole, and the factor may be lowered when additional data strongly suggests safety in the target species as a whole.

In settings involving previously untested chemicals, the adjustment factor is often based on a factor obtained by analysis of other chemicals that may or may not be representative of the chemical currently being examined. Thus, misassignment of an adjustment factor to particular circumstances of the population or chemical involved in the particular scale-up, or any combination thereof, may likely take place.

Overestimating or underestimating the “true” adjustment factors can be costly when developing either safety or efficacy thresholds. For example, in drug safety threshold determination, underestimating the adjustment factor can lead to adverse effects seen in the first-in-human clinical trial, which may prevent an otherwise beneficial drug from progressing any further in drug development. In contrast, in the case where the minimum efficacious dose is much greater than the dose determined for the recommended MRSD, overestimating the adjustment factor may incur significant cost and inefficiency by requiring greater resources to continue clinical trials. In industrial chemical safety threshold determination, if the adjustment factor is underestimated, there is a high probability of harmful exposure in humans in the absence of “clinical trials” for these chemicals. On the other hand, disqualification of what may otherwise be a helpful chemical in an industrial process may occur if the adjustment factor is overestimated.

There are analogous losses involved in over- or underestimating the adjustment factor for determining efficacy threshold. Overestimation may produce an adverse effect, while underestimation may lead to selection of a less beneficial dose than what the target species may be able to receive.

Therefore, an adjustment factor that better accounts for the diversity inherent in the target population could be very beneficial for determining safe and efficacious threshold levels.

### SUMMARY OF THE INVENTION

The present application provides in vitro methods of establishing, or estimating, the distribution of doses, concentrations, or exposure of a chemical or biological agent that produce a threshold level of effect in a plurality of members of a species. The methods include the steps of: a) selecting an effect of interest that occurs in a test species in response to a chemical or biological agent, and that a researcher desires to understand in a target species; b) determining the doses, concentration, or exposure to the chemical or biological agent that result in a threshold level of the effect in that test species; c) using a method of dose extrapolation to estimate the dose, concentration, or exposure that will produce the equivalent threshold level of effect in a reference standard member of the target species; d) selecting an in vitro assay that can be conducted on tissues originated from, or derived from tissues originated from, members of the target species, and that quantitatively measures endpoints that are analogous to, or correlated with, the effect of interest in the target species; e) conducting the selected assay on the reference standard member(s) of the target species using the dose, concentration, or exposure from step c); f) measuring the endpoint(s) and determining a threshold score for that endpoint for the reference standard member; g) selecting a plurality of additional members of the target species; and h) conducting the selected assay on the tissues originated from or derived from tissues originated from each of those additional members, using the doses, concentrations, or exposure of the chemical or biologic agent as necessary to produce the same (or approximate) endpoint score(s) on those members as was previously determined to be the threshold score for the reference standard member in step f) in order to determine the dose, concentration, or exposure of the chemical or biologic agent that produces approximately the same desired endpoint as the reference standard member determined in step f).

### BRIEF DESCRIPTION OF THE FIGURES

Non-limiting embodiments of the methods of the invention are exemplified in the following figures.

FIG. 1 is a graph showing concentration response curves for the 19 cell lines in Table 1. The concentration of the test compound used is shown on the x axis, and the cell proliferation measured as a percentage of the control cell line is shown on the y axis. The x axis is shown in logarithmic scale.

FIG. 2 is a graph showing concentration response curves for 14 hepatocyte cell lines. The concentration of the test compound used (Compound B) is shown on the x axis, and the endpoint of the assay is shown on the y axis. The X axis is expressed in arithmetic scale.

### DETAILED DESCRIPTION

Methods are provided for establishing, or estimating, a threshold limit for the members of a species (referred to herein as the target species) when exposed to a chemical or biological agent (e.g., maximum safe exposure to a toxic agent, or minimum effective dose for a pharmaceutical), taking into account the potential for variation in responses among the members of that species. The method is particularly useful under circumstances wherein that variation in responsiveness cannot scientifically or ethically be ascertained by directly exposing live members of the target species to the agent.

The methods described herein improve on the current practice in the fields of drug development and chemical toxicology of ascertaining a threshold limit (in the case of toxicology, the maximum safe exposure) in another species (the test species), and using a point-estimate dose (or exposure) extrapolation formula to determine a single estimated threshold limit in the target species—one which is implicitly associated with a defined “representative” member of the target species.

The methods provided herein involve conducting in vitro experiments of relevant assays on tissues or cells derived from a plurality of members of the target species to establish a distribution of the doses/concentrations/exposures that lead to the same measured impact or effect as the “representative” member experiences when exposed to the threshold limit dose/concentration/exposure that is currently calculated under the current practice.

The claimed methods may be better understood based on the following detailed description.

### Definitions

The following terms are herein defined as they are used in this application:

As used in this specification, the singular forms “a”, “an” and “the” include the plural referents unless expressly and unequivocally limited to one referent.

“Threshold limit” is used herein as a defined dose, concentration, or exposure at which a response of interest by an individual test subject, or by a reference member of a species or population of interest, changes in a meaningful way. For example, a “threshold” could refer to the exposure at which a reaction initiates or terminates. It could further refer to the exposure that is deemed to be the “maximum safe” dose, concentration, or exposure, as defined by the member's biological reaction remaining below a level known to be harmful to the subject. Conversely, it could refer to the “minimum effective” dose, concentration, or exposure in the case of compounds that are deemed to be beneficial. The terms “dose”, “exposure” and “concentration” are interchangeable herein.

“Exposure”, “dose” and “dose concentration” may be used interchangeably herein. Each may refers to a single exposure, repeated exposure, or continuous exposure.

“Reference standard member” refers to the real or imaginary member of a species whose numerical score on some structural or behavioral measure is deemed to be representative (however the term “representative” may be defined by the researcher) of the population or any sub-population of the species. Representativeness may be as a result of any numerical attribute or behavior or reaction of the member being the median, mean, mode, or some other measure of centrality, or representativeness of the member may be asserted based on one or more factors that are exogenous to the experiment(s) being conducted This reference standard of the target species may also be associated with the minimum, maximum, or any hierarchical response of the members of the species, as well as a centrality or hierarchical response of a plurality within the species. Different reasons may arise for choosing any members of the target population to whom the scale-up most closely applies. For example, choosing the member of the target species with the most robust response in safety threshold determination would lower the risk for an adverse effect occurring later, when the general population is exposed to the agent of interest.

“Endpoint” refers to a quantitatively measurable parameter that is associated with the nature of the effect or reaction of interest. A “desired endpoint” refers to the threshold limit or the measured parameter for the desired effect or reaction of interest.

“Target species” is used herein as the species whose reaction in vivo to the biological or chemical agent being studied is the ultimate objective of the experiments being undertaken. An exemplary target species is a human.

“Biological agent or chemical agent” includes, but are not limited to, industrial chemicals, pharmaceutical agents, bacteria, virus or other disease producing agents, radiation agents and aesthetic or cosmetic agents.

“Test species” as used herein is any species other than the target species on which experiments are conducted for the purpose of directly or indirectly estimating effects on or reactions by the target species. Exemplary test species include animal models, such as laboratory rats, having a reaction to the biological or chemical agent of interest that is biologically similar to the reaction to the same biological or chemical agent by the target species.

“Target Species Equivalent Dose” is used herein to refer to the dose that, when applied to the reference standard member of the target species, is estimated to cause the same degree of effect on a particular endpoint in the reference standard member of the target species as the dose that caused the threshold limit effect on the analogous endpoint in the test species.

“Tissues” or “cells” as used herein refer to any cells, tissues, organs or systems used in the in vitro tests described herein, including but not limited to cells or cell constructs derived from cells or tissues taken from a donor animal, including any cells that are created or maintained external to the animal, but which have the same DNA profile as the animal of interest. For example, “tissues”, as used herein, includes biological samples derived directly or indirectly from biological or bodily fluids such as, but not limited to, blood, urine, cord blood and the like, as well as groups of similar cells from the same origin that carry out the same function together, such as conventional tissue samples. And the term “cells”, as used herein, includes, but is not limited to, human and animal cells including stem cells such as induced pluripotent stem cells (iPSCs), embryonic stem cells and cells derived therefrom.

### DETAILED DESCRIPTION

Platforms and methods for using in vitro experiments to establish a threshold dose, exposure, or concentration limit for a compound in a target species are described herein, wherein an adjustment factor is established for a target species whose threshold level of effect was previously obtained from experimentation on a test species or multiple test species.

Initially, a traditional cross-species dose/concentration/exposure study of the impact of the agent is performed, and the dose is scaled through traditional dose extrapolation methods to the target species, using protocols well familiar to those skilled in the art. This traditional study includes identifying the chemical or biological agent of interest, the target species, the nature of the reaction to the agent that is of interest, and the test species. Then a member or members of the test species are exposed to the agent in whatever doses, concentrations, or exposure and in whatever manner (e.g., oral ingestion, inhalation, injection) are appropriate for the experiment. The effects on the test specimens are measured, and the results are analyzed to determine the threshold effect levels and the associated dose, concentration, or exposure levels. Finally, any known methods for dose extrapolation are used to determine the equivalent dose (sometimes referred to as the Target Species Equivalent Dose or TSED) in the target species.

The present methods include the following platforms and methodologies:

(1) A researcher selects an in vitro test or series of tests that (a) utilize cells or tissues or cells derived from cells that originate from members of the target species and (b) has/have been demonstrated to exhibit reactions in vitro that are highly correlated with in vivo reactions, or endpoints, in the target species which are, to a meaningful degree, analogous to the reactions of interest observed in the test species, as described above. For example, cardiac mitochondria damage in the test population may prompt the researcher to choose a cardiomyocyte cell health assay with a mitochondria health dye to perform on cardiomyocytes from the target population.

In the case that there is not an in vitro test for the identical effects observed in the in vivo test experiments, the researcher may choose another in vitro test as a model for the biological effect that the researcher is trying to either guard against or promote. For example, the presence of a heart attack in the test population may prompt the researcher to do an in vitro test that shows correlation in vitro with heart failure in the target species (e.g., humans), such as a cardiomyocyte cell health assay. Alternative health assays to be used in the method described herein include, but are not limited to, pluripotent stem cell health assays, cardiomyocyte health assay, hepatocyte health assays, neurocyte health assays, respiratory epithelium health assays, embryoid body health assays and any other cell health assays known to those skilled in the art.

(2) The researcher applies the previously determined Target Species Equivalent Dose (TSED) of the agent to cells or tissues originated from (or derived from cells originated from) a plurality of the members of a target species, separately for each donor (at least 5, preferably 20 or more, and most preferably 50 or more).

(3) The researcher arrays the numerical results from (2) in ascending order and selects the member or members to be the “reference standard member” Using any method previously described. The researcher then notes the numerical result(s) of the end points of the test for the reference standard member.

(4) The researcher conducts the in vitro test on some, most, or all of the members of the plurality of the target species as described in (2) at various dose levels surrounding (on one side or both) the TSED utilized in (2).

(5) The researcher may then produce dose response curves for each member so tested. The researcher then calculates the dose for each donor that produces the same numeric result on a particular endpoint as produced by the reference standard member at the TSED utilized in (2). In one embodiment, the dose assigned to each member of the remainder of the population is the one eliciting the effect closest to the threshold effect experienced by the reference standard member. In the case that the finite plurality of doses chosen may not produce effects in the remainder of the target population that are deemed to be sufficiently similar to the threshold effect of the reference standard member, the dose assigned to each member of the remainder of the population may be interpolated between the dose where the effect comes closest to, but does not go past, the threshold effect, and the lowest dose that passes the threshold effect. Note that the interpolation may be linear, geometric, or by any other method. In yet another case, the dose is inferred from nearby doses, without explicit numerical calculation.

(6) The researcher may then develop a distribution of the data that communicates the frequency in which various dose ranges produce the same or interpolated numeric result as the reference standard member produced under the TSED.

Importantly, the current invention also encompasses instances wherein multiple effects in the original test species are of interest, as well as instances in which multiple effects in the target species are of interest because each of them correlates in some way with any one effect of interest in the test species. Further, the current invention encompasses instances wherein multiple endpoints of multiple in vitro tests are of interest because they have correlation with any one in vivo effect in either the test species, the target species, or both.

In converting any numerical results of multiple endpoints to the dose that causes a particular numerical result, the researcher may utilize any number of methods of combining these endpoints, including, but not limited to, overlaps, using the broadest ranges of doses indicated by considering each endpoint separately, unions of the dose ranges, intersections, etc.

(7) The distribution may then be used as an end product (such as for study or documenting the variation in expected responses), or it may be utilized to estimate a preferred dose or exposure threshold limit in the target species. Where it is used to estimate a preferred dose or exposure threshold limit, the researcher uses the distribution created in (6) to identify the in vitro dose that best achieves the researcher's objective in her preferred portion of the population, as modeled by the sample of individuals (e.g., in the case of toxicity, choosing the maximum dose that yields a toxic effect below the acceptable threshold in at least 95 percent of the sample) The researcher then converts the chosen in vitro dose to an in vivo dose using methods known to those skilled in the art.

### EXAMPLES

The present invention may be better understood by reference to the following non-limiting examples.

### Example 1: Determining Toxicity Levels for a Pharmaceutical Compound

Early in the development program for a new pharmaceutical compound—“Compound A”—it was discovered that the likely minimum dose required for the compound to be effective in humans was likely to be one that would produce dose concentration in the blood on the order of 0.002 micromolar. There was some concern as to whether this dose could be tolerated without toxic side effects.

Tests in rats determined that a toxic effect occurred at low doses, namely a severe decline in the rate of cell proliferation in the animal's bone marrow, leading to deleterious decline in the red blood cell count. The NOAEL dose (when the compound was administered orally to the rats) for this effect in rats was measured at 0.0146 mg.

Using a simple (and publicly available) allometric scaling algorithm, the NOAEL dose in rats was translated into a dose (again when administered orally) in the “reference” human being of 1 mg. Using simple tests and standard techniques, this dose was determined to correspond to a dose concentration level in the blood of a “reference” human of about 0.005 micromolar (i.e. doses less than ones that produce a blood concentration below 0.005 appeared safe) Thus, it appeared that the reference human could tolerate the minimum effective dose (associated with a dose concentration of 0.002 micromolar), as this level was below the threshold dose.

However, there was a concern about the potential toxic effects on any humans that might have lower tolerance thresholds than the “reference” human. To examine this question, a series of experiments were conducted to measure the reduction caused by Compound A in the cell proliferation rates of induced pluripotent stem cells (iPSCs) derived from 19 human donors who represented a genetically diverse sample of the population. The Cyquant® assay from Life Technologies was selected as the assay, and the experiments were conducted in accordance with that assay's standard protocol.

Accordingly, iPSCs from the 20 cell lines and a control cell line were plated in multiple replicates into 96-well plates (with extra wells provided for each cell line to serve as controls). Compound A was diluted in media until the dose concentration equaled the 0.005 micromolar level predicted in the allometric scaling calculations described above. The compound was added to the experimental replicate wells, and vehicle only was added to the control wells. After incubation for 48 hours under conditions to allow replication, the results of the experimental replicate wells were measured and analyzed, and the results were then compared to the results from the vehicle control wells. The resulting endpoint consisted of a measure of the proliferation rate of each iPSC line under challenge from a 0.005 micromolar dose concentration of Compound A, expressed as percentage of the proliferation rate of that same cell line challenged only by the vehicle control. Results for 19 cell lines (after the removal of one cell line which failed to show results due to an experimental error) are shown in Table 1 below.

TABLE 1

| | Proliferation (Percent of |
|---|---|
| Donor | Control) at 0.005 μM |
| # | concentration |
| 1 | 26.11% |
| 2 | 28.34% |
| 3 | 30.79% |
| 4 | 31.71% |
| 5 | 36.52% |
| 6 | 38.65% |
| 7 | 40.85% |
| 8 | 43.57% |
| 9 | 50.46% |
| 10 | 59.72% |
| 11 | 60.30% |
| 12 | 60.40% |
| 13 | 60.50% |
| 14 | 63.30% |
| 15 | 74.14% |
| 16 | 75.83% |
| 17 | 77.38% |
| 18 | 82.41% |
| 19 | 102.99% |

The cell line that produced the median result was Cell Line 10. Therefore, Cell Line 10 was designated as the “representative” human whose tolerance was estimated via the dose extrapolation analysis above. Because the “proliferation score” (percent of control) for Cell Line 10 was 60%, a presumption was adopted that the threshold level of reduction in cell proliferation that humans could tolerate was that which corresponded to a score of 60% in the Cyquant® test.

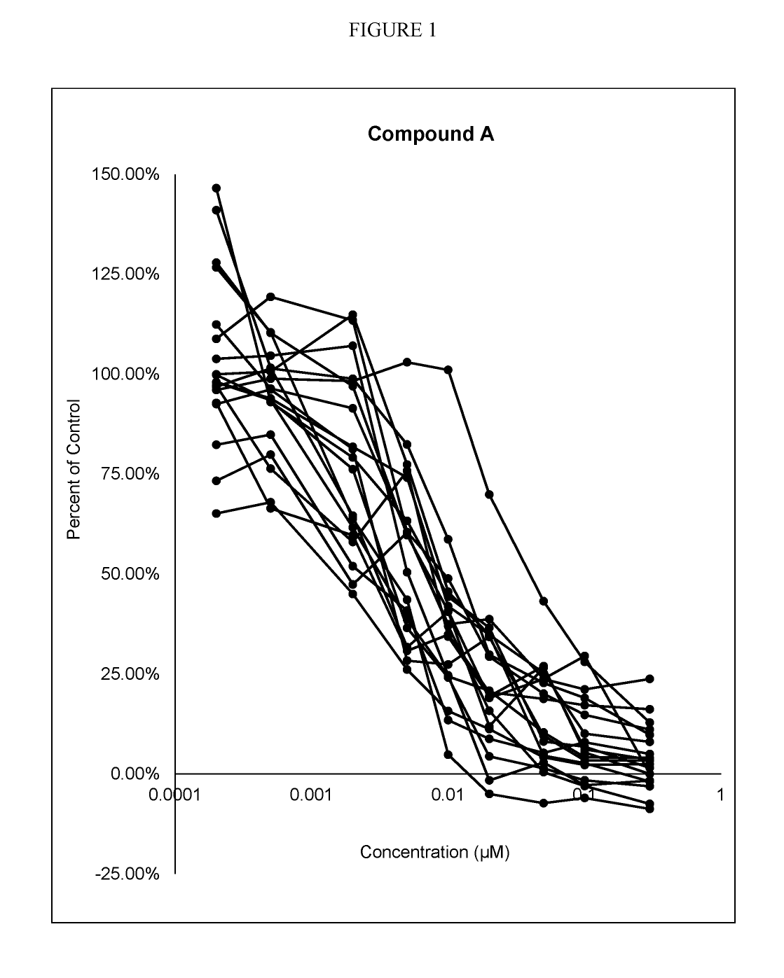

The dose concentrations that produced a proliferation score of 60% in the cell lines from the other 18 donors were then determined. Although other methods could have been used, these doses were determined by developing full dose-response curves as shown in FIG. 1. From this figure, the threshold dose concentrations were calculated and are shown in Table 2 below. Four of the 18 lines (19 lines when Cell Line 10 is included) or 21% of the lines, fell below a dose concentration of 0.002 micromolar (i.e., the dose concentration corresponding to the estimated minimum effective dose).

TABLE 2

| Donor | Concentration to Constrain |
|---|---|
| # | Proliferation to 60% (μM) |
| 1 | 0.00080 |
| 12 | 0.00117 |
| 7 | 0.00143 |
| 16 | 0.00170 |
| 3 | 0.00183 |
| 6 | 0.00214 |
| 4 | 0.00227 |
| 8 | 0.00237 |
| 2 | 0.00289 |
| 5 | 0.00290 |
| 9 | 0.00428 |
| 10 | 0.00490 |
| 11 | 0.00498 |
| 13 | 0.00501 |
| 14 | 0.00565 |
| 15 | 0.00680 |
| 17 | 0.00730 |
| 18 | 0.00960 |
| 19 | 0.02810 |

Thus, the potential for an adverse toxicity finding in late stage clinical trials was high. Compound A might pass Phase I trials, because conventional safety margins of 10× (pharmacokinetic and pharmacodynamic effects combined) would dictate that the dosing in Phase I start at 0.0005 micromolar (i.e., one-tenth of the dose indicated by the allometric scaling process), below the lowest threshold dose found among the 19 cell lines (i.e., 0.00075 micromolar). However, given the significant portion of lines tested whose threshold fell below the minimum effective dose, Phase II would likely reveal toxicity effects in some trial participants at a lower dose than the dose at which the compound would produce benefits. As a result, it was recommended that development of Compound A be curtailed.

### Example 2: Assisting the Choice of Suspension Vehicle for a Promising Compound

A pharmaceutical company has developed a compound (Compound B) that looks promising through the early (pre-clinical) phases of drug development. This compound must be administered to the patient in suspension form (i.e. wherein the molecules of the compound are dissolved in a liquid that holds the molecules in suspension at the time when a single dose is drawn from a larger batch and administered to the patient).

The time has come in the process to identify a suitable vehicle to fulfill the suspension function. A number of attributes distinguish the various candidates vehicles (such as relative cost, longevity, etc.), but the first criterion is the ability of a candidate vehicle to carry a high enough concentration of the compound (without the molecules “dropping out” of suspension) to produce a beneficial effect for a large portion of the population (in this, case 75 percent of the population or higher).

A researcher recognizes the criterion as including two sub-issues: (1) “What is the minimum concentration of Compound B necessary to achieve a beneficial effect in various portions of the population?” and (2) “What is the maximum concentration of Compound B that a candidate vehicle can successfully suspend for the required duration?” The researcher uses the present invention to address the first of these two sub-issues.

Previous to the experiments in question, the pharmaceutical company has conducted in vivo studies on dogs to establish the Minimally Effective Dose (MED) concentration in dogs, and used a typical allometric dose extrapolation formula to estimate the MED for a gender and weight defined reference standard human (i.e. a generalized 60 kg male)—a resulted in a blood dose concentration level of 13.4 micromolar. However, the pharmaceutical company recognizes that humans vary in their responsiveness to compounds, and therefore recognizes that this dose may or may not be sufficient to provide benefits to some, most or almost all of the population. Therefore, the researcher is tasked with finding the estimated minimum dose (or dose concentration, since the latter can be translated to the former) of Compound B required to achieve a minimum level of beneficial impact in 75 percent of the population.

Because the beneficial effect is associated with an increase in the production of a particular protein in the liver, the researcher selects an in vitro assay that detects the production of that particular protein by hepatocytes. The researcher then obtains a sample of iPSCs from a cross-section (as defined by gender, race and weight) of 14 healthy adults, one of which (a male weighing close to 60 kg) is selected as the reference standard human.

The researcher differentiates the iPSCs from all of the donors into hepatocytes, following the protocol from, and using the materials of, the Cellartis Definitive Endoderm Differentiation Kit and the Cellartis Hepatocyte Differentiation Kit, available from Clontech Laboratories, Inc., of Mountain View, Calif.

The researcher then conducts the assay of Compound B on the cells from the reference standard human at the previously determined MED dose concentration of 13.4 micromolar, as well as two dose concentrations that surround the MED-3.6 micromolar and 50.0 micromolar. The assay conducted with the MED results in a score of 56 percent on the chosen endpoint.

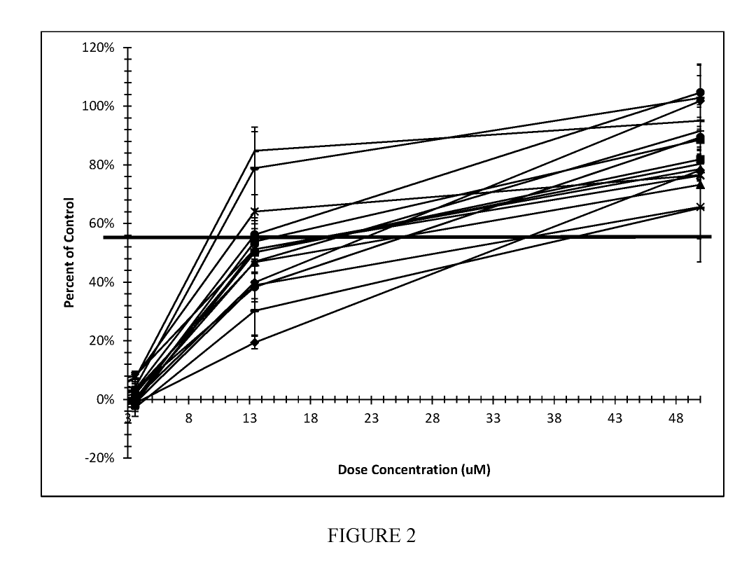

The researcher then repeats the assay on the other members of the sample using the same dose concentrations as above. The results are shown in FIG. 2. The researcher then interpolates the dose concentrations for the other 13 donors that would have resulted in the same 56 percent score as was exhibited by the standard reference human at the MED. The results are shown in Table 3 below.

TABLE 3

| Observation | Dose Concentration |
|---|---|
| (ranked lowest to highest) | (micromolar) |
| 1 | 9.6 |
| 2 | 10.4 |
| 3 | 11.9 |
| 4 | 13.4 |
| 5 | 15.7 |
| 6 | 19.4 |
| 7 | 19.7 |
| 8 | 20.7 |
| 9 | 23.0 |
| 10 | 25.9 |
| 11 | 26.4 |
| 12 | 36.2 |
| 13 | 37.2 |
| 14 | 40.5 |

The 75th percentile in a sample of 14 occurs between the 10th observation and the 11th observation. Therefore the researcher identifies the dose associated with the 11th observation, i.e. 26.4 micromolar, as the estimated dose concentration required to produce a minimum beneficial effect in at least 75 percent of the population.

As a result, the pharmaceutical company narrows the list of candidate vehicles to those that can successfully suspend a dose concentration of 26.4 micromolar of Compound B.

1. An in vitro method for estimating a distribution of exposures to a chemical or biological agent that produce a threshold level of effect among members of a plurality of a target species, comprising:
a) identifying an effect of interest that occurs in both a test species and in a target species in response to exposure to the agent;
b) determining the amount of exposure to the agent that results in a point-estimate of the threshold limit of effect in the test species;
c) extrapolating the amount of exposure that will produce the equivalent threshold limit of effect in a reference standard member of the target species to form a Target Species Equivalent Dose (TSED);
d) conducting an in vitro assay on tissues or cells from, or derived from tissues or cells from, a sample of a reference standard member of the target species using the exposure equivalent to the TSED that is determined to be the appropriate exposure for the assay;
e) measuring an endpoint effect and determining the score for that endpoint with respect to the reference standard member; and
f) conducting the in vitro assay on tissues or cells from, or derived from tissues or cells from, a sample of additional members of the target species using various exposures as necessary to estimate for each additional member sample the exposure that produces in that member the same score as was obtained in e) for the reference standard member.

2. The method of claim 1, wherein the agent is an industrial chemical, a pharmaceutical agent, bacteria, virus or other disease producing agent, radiation agent or aesthetic or cosmetic agent.

3. The method of claim 1, wherein the agent is a tear agent, vomiting agent, malodorant, incapacitating agent, blister agent, nerve agent, vesicant agent, urticant, or choking agent.

4. The method of claim 1, wherein the assay is a pluripotent stem cell assay, a cardiomyocyte assay, a hepatocyte assay, a neurocyte assay, a respiratory epithelium assay or an embryoid body assay.

5. The method of claim 1, wherein the cells are induced pluripotent stem cells, or cells or tissues derived directly or indirectly from pluripotent stem cells.

6. The method of claim 1, wherein the desired endpoint is the maximum exposure at which a toxic effect is predicted to not occur.

7. The method of claim 1, wherein the desired endpoint is the minimum exposure at which a desired beneficial effect is predicted to occur.

8. An in vitro method for estimating the distribution of exposures to a chemical or biological agent that produce a threshold level of toxic effect among the members of a plurality of a target species, comprising:
a) identifying a toxic effect of interest that occurs in both a test species and in a target species in response to exposure to the agent;
b) determining the amount of exposure to the agent that results in a point-estimate of the threshold limit of toxic effect in the test species;
c) extrapolating the amount of exposure that will produce the equivalent threshold limit of effect in a reference standard member of the target species to form a Target Species Equivalent Dose (TSED);
d) conducting an in vitro assay on tissues or cells from, or derived from tissues or cells from, a sample of a reference standard member of the target species using the exposure that is equivalent to the TSED that is determined to be the appropriate exposure for the assay,
e) measuring an endpoint effect and determining the score for that endpoint with respect to the reference standard member; wherein the score corresponds to the maximum exposure at which a toxic effect is not detected; and
f) conducting the in vitro assay on tissues or cells from, or derived from tissues or cells from, a sample of additional members of the target species using various exposures as necessary to estimate for each additional member sample the exposure that produces in that member the same score as was obtained in e) for the reference standard member.

9. The method of claim 8, wherein the agent is an industrial chemical, a pharmaceutical agent, bacteria, virus or other disease producing agent, radiation agent or aesthetic or cosmetic agent.

10. The method of claim 8, wherein the agent is a tear agent, vomiting agent, malodorant, incapacitating agent, blister agent, nerve agent, vesicant agent, urticant, or choking agent.

11. The method of claim 8, wherein the assay is a pluripotent stem cell assay, a cardiomyocyte assay, a hepatocyte assay, a neurocyte assay, a respiratory epithelium assay or an embryoid body assay.

12. The method of claim 8, wherein the cells are induced pluripotent stem cells, or cells or tissues derived directly or indirectly from pluripotent stem cells.

13. An in vitro method for estimating the distribution of exposures to a chemical or biological agent that produce a threshold level of beneficial effect among the members of a plurality of a target species, comprising:
a) identifying a beneficial effect of interest that occurs in both a test species and in a target species in response to exposure to the agent;
b) determining the amount of exposure to the agent that results in a point-estimate of the threshold limit of beneficial effect in the test species;
c) extrapolating the amount of exposure that will produce the equivalent threshold limit of effect in a reference standard member of the target species to form a Target Species Equivalent Dose (TSED);
d) conducting an in vitro assay on tissues or cells from, or derived from tissues or cells from, a sample of a reference standard member of the target species using the exposure that is equivalent to the TSED that is determined to be the appropriate exposure for the assay,
e) measuring an endpoint effect and determining the score for that endpoint with respect to the reference standard member; wherein the score corresponds to the minimum exposure at which a beneficial effect is detected; and
f) conducting the in vitro assay on tissues or cells from, or derived from tissues or cells from, a sample of additional members of the target species using various exposures as necessary to estimate for each additional member sample the exposure that produces in that member the same score as was obtained in e) for the reference standard member.

14. The method of claim 13, wherein the agent is a pharmaceutical agent, aesthetic agent or cosmetic agent.

15. The method of claim 13, wherein the assay is a pluripotent stem cell assay, a cardiomyocyte assay, a hepatocyte assay, a neurocyte assay, a respiratory epithelium assay or an embryoid body assay.

16. The method of claim 13, wherein the cells are induced pluripotent stem cells, or cells or tissues derived directly or indirectly from pluripotent stem cells.
